(19) United States
(12) Patent Application Publication
(10) Pub. No.: US 2017/0055957 A1
(43) Pub. Date: Mar. 2, 2017

(54) ULTRASONIC DIAGNOSTIC DEVICE AND PROGRAM

(71) Applicant: GE MEDICAL SYSTEMS GLOBAL TECHNOLOGY COMPANY, LLC, Waukesha, WI (US)

(72) Inventors: Shunichiro Tanigawa, Hino-Shi (JP); Atsuko Matsunaga, Hino-shi (JP)

(21) Appl. No.: 15/120,039

(22) PCT Filed: Feb. 18, 2015
(86) PCT No.: PCT/US15/16316

§ 371 Date: Aug. 18, 2016

(30) Foreign Application Priority Data
Feb. 19, 2014 (JP) 2014-029629

(51) Int. Cl. A61B 8/00; A61B 8/08

(52) U.S. Cl. CPC A61B 8/54; A61B 8/5207; A61B 8/485; A61B 8/461; A61B 8/469

(57) ABSTRACT

An ultrasonic diagnostic device capable of preventing useless transmission of an ultrasonic pulse for detection from being performed characterized by including a processor that executes a program for controlling an ultrasonic probe such that transmission of an ultrasonic push pulse to a biological tissue of a test object and transmission of a plurality of ultrasonic pulses for detection on the same sound ray in order to detect a shear wave generated in the aforementioned biological tissue with the push pulse concerned are alternatively repeated, the program for controlling the aforementioned ultrasonic probe such that the aforementioned ultrasonic pulses for detection are transmitted on the aforementioned sound ray until the aforementioned shear wave is detected at a predetermined number of detection points on the sound ray that the aforementioned ultrasonic pulses for detection are transmitted.

### CROSS-REFERENCE TO RELATED APPLICATIONS

This application is a filing under 35 U.S.C. 371 of international application number PCT/U.S. 2015/016316, filed Feb. 18, 2015, which claims priority to Japan application number 2014-029629, filed Feb. 19, 2014, the entire disclosure of each of which is hereby incorporated by reference.

### BACKGROUND

The present invention relates to an ultrasonic diagnostic device and a program adapted to transmit an ultrasonic pulse for detection for detecting a shear wave generated in a biological tissue with an ultrasonic push pulse.

An elasticity measurement technique for transmitting an ultrasonic pulse (a push pulse) that is high in sound pressure from an ultrasonic probe and measuring an elasticity of the biological tissue is known (see, for example, Patent Document 1). More specifically, the shear wave generated in the biological tissue with the push pulse is detected with the ultrasonic pulse for detection and a propagation velocity of the shear wave and an elasticity value of the biological tissue are calculated. Then, an elastic image having a color and so forth according to a calculated value is displayed.

### SUMMARY OF THE INVENTION

Here, in a case where a two-dimensional elastic image is to be displayed, transmission/reception of the ultrasonic pulses for detection for a plurality of sound rays is performed in a two-dimensional region of interest that the elastic image is to be displayed. However, there are cases when it is difficult to detect the shear waves on all of the sound rays in the two-dimensional region of interest by one-time transmission of the push pulse. Thus, in order to obtain the elastic images of one frame, the shear waves are detected with respect to all of the sound rays in the aforementioned region of interest by alternately repeating transmission of the push pulse and transmission/reception of the ultrasonic pulse for detection.

In addition, in order to cause the two-dimensional elastic image to be displayed, it is necessary to detect the shear wave at a plurality of points on one sound ray. Thus, transmission/reception of the ultrasonic pulse for detection is being performed a plurality times on the same sound ray.

The number of times of transmission/reception of the ultrasonic pulse for detection on one sound ray is a fixed value that has been set in advance. Therefore, even after the shear wave has been detected at all of the points on one sound ray, transmission/reception of the ultrasonic pulses for detection is performed in some cases. in this case, this means that transmission/reception of the ultrasonic pulses for detection that is useless acoustically and temporally is being performed.

The invention of one viewpoint that has been made in order to solve the above-mentioned problems is an ultrasonic diagnostic device characterized by including a processor that executes a program for controlling an ultrasonic probe such that transmission of an ultrasonic push pulse to a biological tissue of a test object and transmission of a plurality of ultrasonic pulses for detection on the same sound ray in order to detect the shear wave generated in the biological tissue with the push pulse concerned are alternatively repeated, the program for controlling the aforementioned ultrasonic probe such that the aforementioned ultrasonic pulses for detection are transmitted on the aforementioned sound ray until the aforementioned shear wave is detected at a predetermined number of detection points on the sound ray that the aforementioned ultrasonic pulses for detection are transmitted.

According to the invention of the above-mentioned one viewpoint, the ultrasonic pulses for detection are transmitted on the aforementioned sound ray until the aforementioned shear wave is detected at the predetermined number of detection points on the sound ray that the aforementioned ultrasonic pulses for detection are transmitted. Therefore, since when the aforementioned shear wave is detected at the predetermined number of detection points on the aforementioned sound ray, transmission of the ultrasonic pulses for detection on the aforementioned sound ray is terminated, performance of useless transmission of the ultrasonic pulses for detection can be prevented.

### BRIEF DESCRIPTION OF THE DRAWINGS

FIG. 1 is a block diagram showing a schematic configuration of an ultrasonic diagnostic device that is one example of an embodiment of the present invention.

FIG. 2 is a block diagram showing a configuration of an echo data processing unit.

FIG. 3 is a block diagram showing a configuration of a display control unit.

FIG. 4 is a diagram showing a display unit that a B-mode image and an elastic image have been displayed.

FIG. 5 is a diagram showing the display unit that a region of interest has been set on the B-mode image.

FIG. 6 is a flowchart showing processing for causing the elastic image to be displayed.

FIG. 7 is a diagram for explaining transmission of a push pulse and a shear wave generated with the push pulse.

FIG. 8 is a diagram for explaining transmission/reception of an ultrasonic pulse for detection corresponding to transmission of the push pulse.

FIG. 9 is a diagram for explaining detection points for the shear wave.

### DETAILED DESCRIPTION

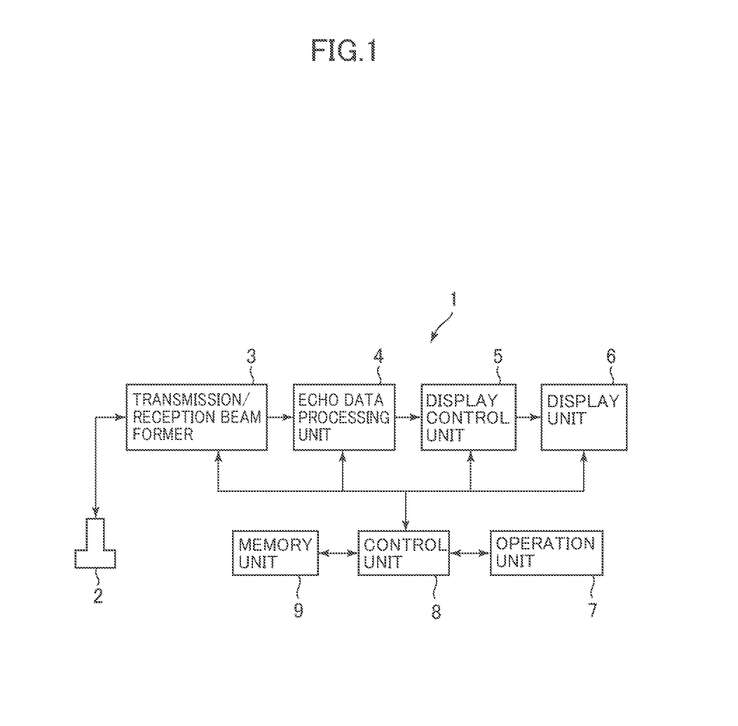

In the following, embodiments of the present invention will be described with reference to the drawings. An ultrasonic diagnostic device 1 shown in FIG. 1 is provided with an ultrasonic probe 2, a transmission/reception beam former 3, an echo data processing unit 4, a display control unit 5, a display unit 6, an operation unit 7, a control unit 8 and a memory unit 9.

The aforementioned ultrasonic probe 2 is one example of an embodiment of an ultrasonic probe in the present invention and transmits an ultrasonic wave to a biological tissue of a test object. An ultrasonic pulse (a push pulse) for making a shear wave generate in the biological tissue is transmitted by this ultrasonic probe 2. In addition, an ultrasonic pulse for detection for detecting the shear wave is transmitted and an echo signal thereof is received by the aforementioned ultrasonic probe 2.

Transmission/reception of the aforementioned ultrasonic wave for detection is performed on a plurality of sound rays in a later described region of interest R. As described later, after the aforementioned push pulse has been transmitted one time, transmission/reception of the aforementioned ultrasonic pulse for detection on one sound ray is performed. Transmission of the aforementioned push pulse and transmission/reception of the aforementioned ultrasonic pulse for detection are alternately repeated. In addition, after the push pulse has been transmitted one time, the aforementioned ultrasonic pulse for detection is transmitted/received a plurality of times on one sound ray.

Further, an ultrasonic pulse for a B-mode image for creating a B-mode image is transmitted and an echo signal thereof is received by the aforementioned ultrasonic probe 2,

The aforementioned transmission/reception beam former 3 drives the aforementioned ultrasonic probe 2 on the basis of a control signal from the aforementioned control unit 8 to make it transmit the aforementioned various ultrasonic pulses having predetermined transmission parameters. In addition, the transmission/reception beam former 3 performs signal processing such as phasing addition processing and so forth in regard to the echo signal of the ultrasonic wave.

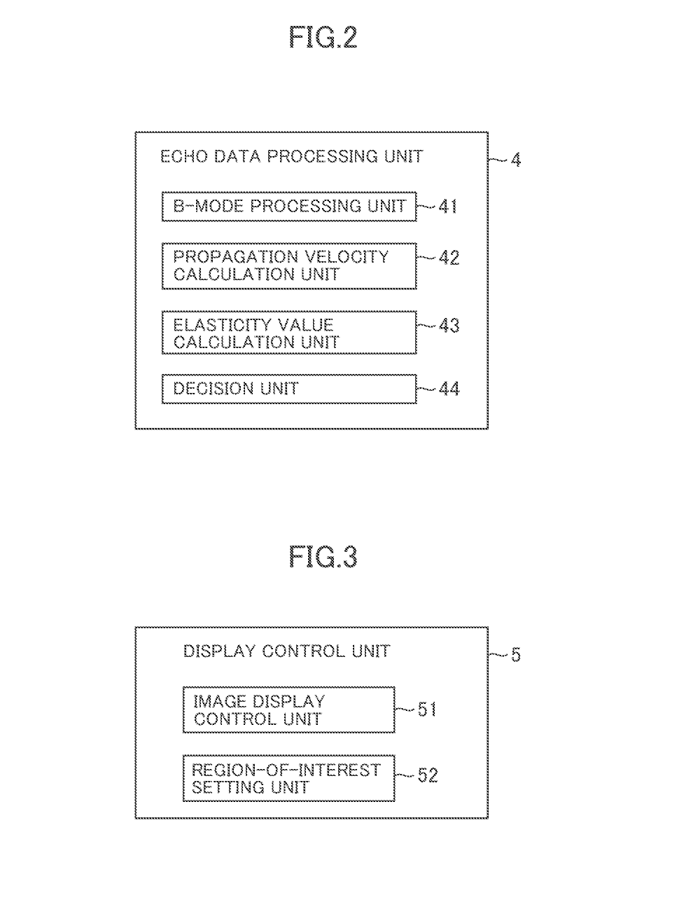

The aforementioned echo data processing unit 4 has a B-mode processing unit 41, a propagation velocity calculation unit 42, an elasticity value calculation unit 43 and a decision unit 44 as shown in FIG. 2. The aforementioned B-mode processing unit 41 performs B-mode processing such as logarithmic compression processing, envelope detection processing and so forth on echo signal that has been output from the aforementioned transmission/reception beam former 3 and creates B-mode data.

In addition, the aforementioned propagation velocity calculation unit 42 calculates the propagation velocity of the aforementioned shear wave on the basis of the echo data of the aforementioned ultrasonic pulse for detection that has been output from the aforementioned transmission/reception beam former 3 (a propagation velocity calculation function). In addition, the aforementioned elasticity value calculation unit 43 calculates the elasticity value of the biological tissue that the push pulse has been transmitted on the basis of the aforementioned propagation velocity (an elasticity value calculation function). Details thereof will be described later. The aforementioned propagation velocity calculation function and the aforementioned elasticity value calculation function are examples of an embodiment of a measured value calculation function in the present invention. In addition, the aforementioned propagation velocity and the aforementioned elasticity value are examples of an embodiment of a measured value relevant to the elasticity of the biological tissue in the present invention.

Incidentally, only the aforementioned propagation velocity may be calculated and the aforementioned elasticity value may not necessarily be calculated. Data on the aforementioned propagation velocity or data on the aforementioned elasticity value will be referred to as elasticity data.

The aforementioned decision unit 44 decides whether the shear waves have been detected at all of the detection points on one sound ray as described later.

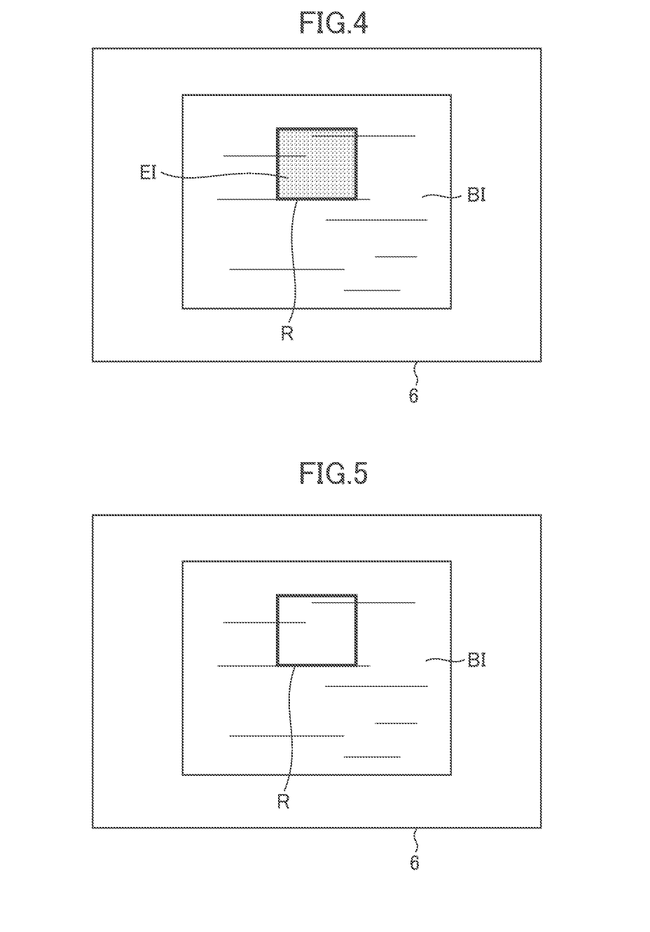

The aforementioned display control unit 5 has an image display control unit 51 and a region-of-interest setting unit 52 as shown in FIG. 3. The aforementioned image display control unit 51 scan-converts the aforementioned B-mode data by a scan converter to create B-mode image data and makes the aforementioned display unit 6 display a B-mode image based on this B-mode image data. In addition, the aforementioned image display control unit 51 scan-converts the aforementioned elasticity data by the scan converter to create elastic image data and makes the aforementioned display unit 6 display an elastic image based on this elastic image data.

As shown in FIG. 4, the aforementioned elastic image EI is a two-dimensional image to be displayed in the region of interest R set on the aforementioned B-mode image BI. The aforementioned elastic image EI is a color image having a color according to the aforementioned propagation velocity or the aforementioned elasticity value. The aforementioned image display control unit 51 synthesizes the aforementioned B-mode image data and the aforementioned elastic image data to create synthetic image data and makes the aforementioned display unit 6 display an image based on this synthetic image data. Therefore, the aforementioned elastic image EI is a translucent image through which the background B-mode image BI transmits.

The aforementioned region of interest R is set by the aforementioned region-of-interest setting unit 52. More specifically, the aforementioned region-of-interest setting unit 52 sets the aforementioned region of interest R on the basis of an input on the aforementioned operation unit 7 by an operator. The aforementioned region of interest R is a transmission/reception region for the aforementioned ultrasonic pulse for detection.

The aforementioned display unit 6 is an LCD (Liquid Crystal Display), an organic EL (Electro-Luminescence) display and so forth. Though not shown in the drawing in particular, the aforementioned operation unit 7 is configured by including a keyboard, a pointing device such as a trackball and so forth and others in order that the operator may input instructions and information.

The aforementioned control unit 8 is a processor such as a CPU (Central Processing Unit) and so forth, This control unit 8 reads out a program stored in the aforementioned memory unit 9 and controls the respective units of the aforementioned ultrasonic diagnostic device 1. For example, the aforementioned control unit 8 reads out the program stored in the aforementioned memory unit 9 and makes it execute functions of the aforementioned transmission/reception beam former 3, the aforementioned echo data processing unit 4 and the aforementioned display control unit 5 in accordance with the read-out program.

The aforementioned control unit 8 may execute all of the functions of the aforementioned transmission/reception beam former 3, all of the functions of the aforementioned echo data processing unit 4 and all functions of the functions of the aforementioned display control unit 5 in accordance with the program or may execute only some functions in accordance with the program. In a case where the aforementioned control unit 8 executes only some functions, the remaining functions may be executed by hardware such as circuits and so forth.

Incidentally, the functions of the aforementioned transmission/reception beam former 3, the aforementioned echo data processing unit 4 and the aforementioned display control unit 5 may be implemented by hardware such as the circuits and so forth.

The aforementioned memory unit 9 is an HDD (Hard Disk Drive), and/or a semiconductor memory such as a RAM (Random Access Memory), and/or a ROM (Read Only Memory) and so forth.

Next, the operation of the ultrasonic diagnostic device 1 of the present example will be described. First, the operator performs transmission/reception of an ultrasonic wave for the B-mode on the test object and causes the B-mode image BI based on the echo signal to be displayed as shown in FIG. 5. Then, he sets the region of interest R on this B-mode image BI. This region of interest R is set in a region where he wishes to display the elastic image.

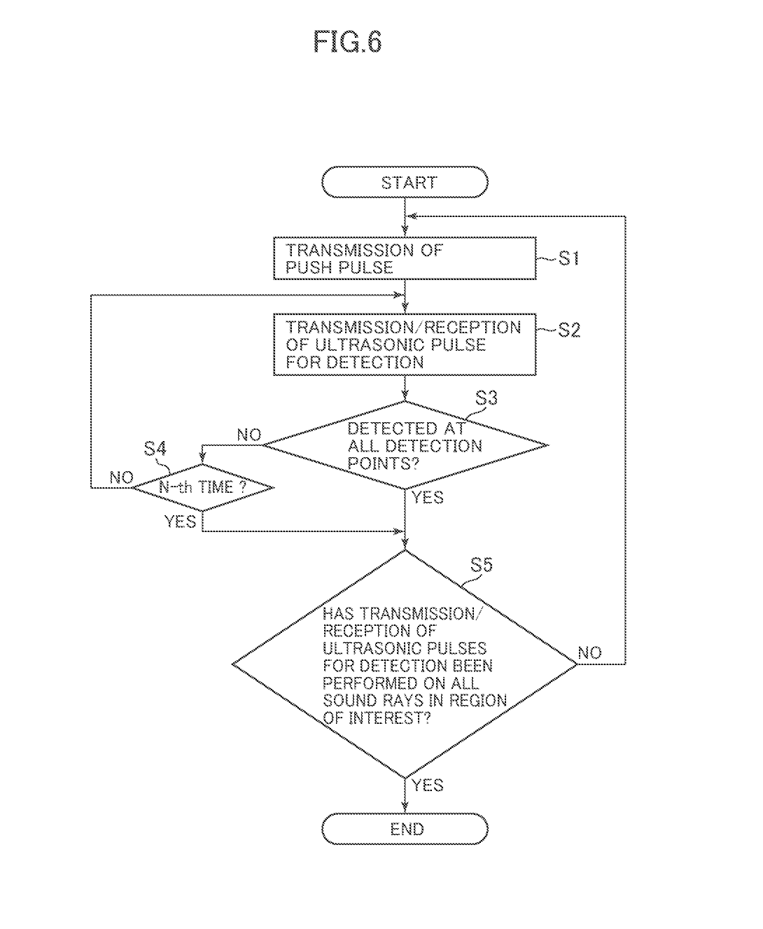

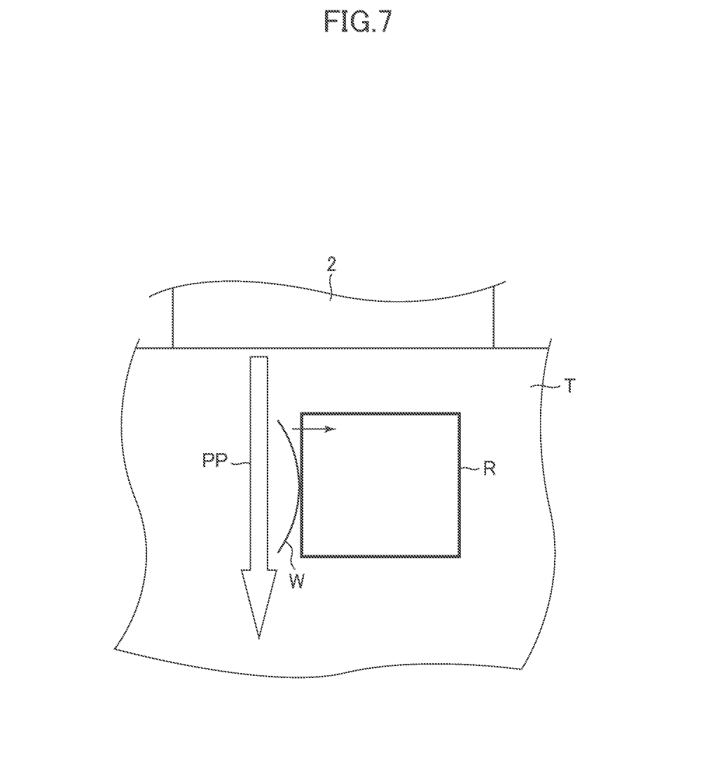

Next, the operator performs an input for causing the elastic image to be displayed by the aforementioned operation unit 7, Processing for causing the elastic image to be displayed will be described on the basis of a flowchart in FIG. 6. When the input for causing the elastic image to be displayed is made by the aforementioned operation unit 7, the processing in FIG. 6 is started.

First, in step S1, the aforementioned control unit 8 causes a push pulse PP to be transmitted from the aforementioned ultrasonic probe 2 to a biological tissue T as shown in FIG. 7. in FIG. 7, the aforementioned push pulse PP is shown by a sound ray (an arrow) (the same shall apply to the succeeding drawings). The aforementioned push pulse PP is transmitted to the outer side of the vicinity of the aforementioned region of interest R. A shear wave W is generated in the biological tissue T with the aforementioned push pulse PP. This shear wave W propagates in the aforementioned biological tissue T in a direction (an arrow direction in FIG. 7) away from the aforementioned push pulse.

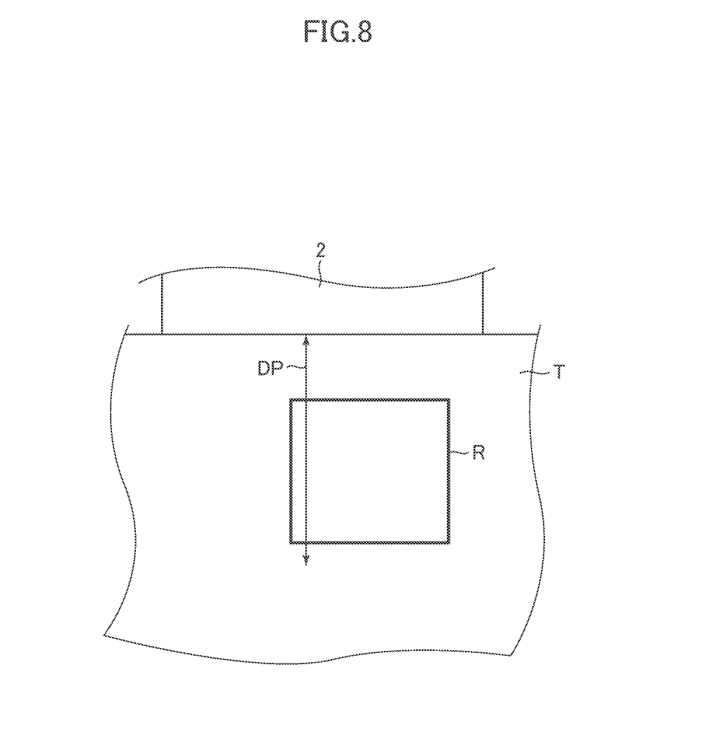

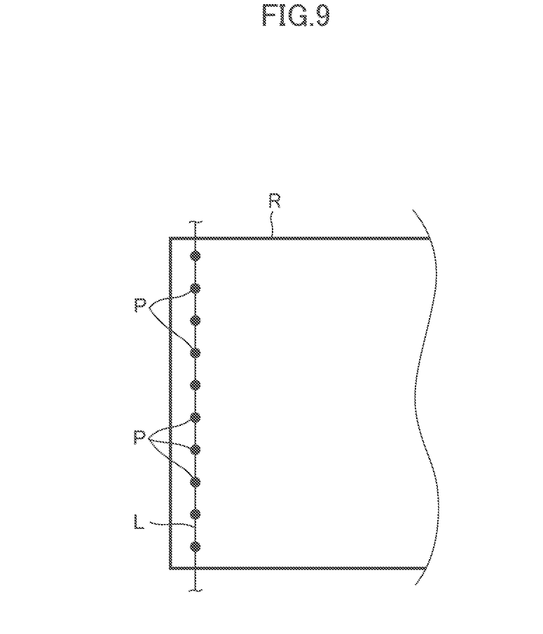

Next, in step S2, the aforementioned control unit 8 causes an ultrasonic pulse for detection DP to be transmitted/received to the aforementioned biological tissue T by the aforementioned ultrasonic probe 2. The aforementioned ultrasonic pulse for detection DP is an ultrasonic pulse for detecting the shear wave W (illustration thereof is omitted in FIG. 8) that propagates in the aforementioned region of interest R. The aforementioned ultrasonic pulse for detection DP is shown by a sound ray (an arrow) in FIG. 8.

Next, in step S3, the aforementioned decision unit 44 decides whether the aforementioned shear wave W has been detected at a predetermined number of detection points on the sound ray of the aforementioned ultrasonic pulse for detection that has been transmitted/received in the aforementioned step S2.

Here, in order to display a two-dimensional elastic image, it is necessary to perform detection of the aforementioned shear wave W at a plurality of detection points P in the region of interest R per one sound ray L. The number of the aforementioned detection points P is set in advance. The aforementioned decision unit 44 decides whether the aforementioned shear wave W has been detected at all of the predetermined number of the aforementioned detection points P in the aforementioned step S3.

In the aforementioned step S3, in a case where it has been decided that the aforementioned shear wave W is not detected at all of the detection points P (“NO” in the aforementioned step S3), it proceeds to the process in step S4. In this step S4, the aforementioned control unit 8 decides whether transmission/reception of the aforementioned ultrasonic pulse for detection DP is the N-th time (N≧2). N is set in advance to a numeral that the aforementioned shear wave W can be detected at all of the detection points P.

In the aforementioned step S4, in a case where it has been decided that it is not the N-th time (“NO” in the aforementioned step S4), it returns to the process in the aforementioned step S2. Thereby, the ultrasonic pulse for detection DP is transmitted to the aforementioned biological tissue T on the same sound ray as that in the last time and the echo signal of this ultrasonic pulse for detection DP is received.

In the aforementioned step S3, in a case where it has been decided that the aforementioned shear wave W has been detected at all of the detection points P (“YES” in the aforementioned step S3), it shifts to the process in step S5. Therefore, transmission/reception of the aforementioned ultrasonic pulse for detection DP will be performed on the same sound ray until the aforementioned shear wave is detected at the predetermined number of the aforementioned detection points P. In addition, in the aforementioned step S4, also in a case where it has been decided that it is the N-th time (“YES” in the aforementioned step S4), it shifts to the process in step S5.

In step S5, the aforementioned control unit 8 decides whether transmission/reception of the aforementioned ultrasonic pulse for detection DP has been performed on all of the sound rays in the aforementioned region of interest R.

In the aforementioned step S5, in a case where it has been decided that transmission/reception of the aforementioned ultrasonic pulse for detection DP is not performed on all of the sound rays in the aforementioned region of interest R (“NO” in the aforementioned step S5), it returns to the process in the aforementioned step S1. Thereby, in the aforementioned step S1, after the push pulse PP has been transmitted again to the aforementioned biological tissue T, the ultrasonic pulse for detection DP is transmitted/received in the aforementioned step S2. However, this ultrasonic pulse for detection DP is transmitted/received on a neighboring sound ray of the sound ray that the last time transmission/reception of the ultrasonic pulse for detection DP has been performed. Incidentally, the aforementioned push pulse PP may be transmitted on the same sound ray as that in the last time and may be transmitted on a sound ray that is different from that in the last time. Then, the processes in the aforementioned steps S3, S4 are performed. From the above, transmission of the aforementioned push pulse PP and transmission/reception of the aforementioned ultrasonic pulse for detection DP will be repeated such that the aforementioned ultrasonic pulses for detection DP are transmitted/received on the different sound rays in the aforementioned region of interest R.

On the other hand, in the aforementioned step S5, in a case where it has been decided that transmission/reception of the aforementioned ultrasonic pulses for detection DP has been performed on all of the sound rays in the aforementioned region of interest R (“YES” in the aforementioned step S5), the processing is terminated. From the above, the echo signals of the ultrasonic pulses for detection DP for creating the elastic image data of one frame are acquired and the elastic image EI is displayed in the aforementioned region of interest R. After that, the frames of the B-mode image BI and the elastic image EI are updated by repeating transmission/reception of the aforementioned ultrasonic wave for the B-mode and the processes in the aforementioned steps S1 to S5.

According to the present example, in the aforementioned step S3, in a case where it has been decided that the aforementioned shear wave W has been detected at all of the detection points on one sound ray, transmission/reception of the ultrasonic pulse for detection DP on that sound ray is terminated. Therefore, unnecessary transmission/reception of the ultrasonic pulse for detection DP is not performed and transmission/reception of the aforementioned ultrasonic pulse for detection DP can be reduced in comparison with a case where transmission/reception of the ultrasonic pulse for detection DP is performed by the number of times that has been set in advance, and therefore the frame rate can be improved.

As mentioned above, although the present invention has been described by the aforementioned embodiments, it goes without saying that the present invention can be variously modified and embodied within a range not altering the gist thereof. It can be similarly applied also to a case where, for example, after the aforementioned push pulse PP has been transmitted one time, the aforementioned ultrasonic pulse for detection DP is transmitted/received the plurality of times on each of the plurality of sound rays.

In addition, after the aforementioned push pulse PP has been transmitted in the aforementioned step S1 and after transmission/reception of the aforementioned ultrasonic pulse for detection DP has been repeated M (M<N) times in the aforementioned step S2, decision in the aforementioned step S3 may be performed. M is set to a numeral of such an extent that the aforementioned shear wave W is not detected at all of the detection points P.

1-10. (canceled)

11. An ultrasonic diagnostic device, characterized by comprising:
a processor that executes a program for controlling an ultrasonic probe such that transmission of an ultrasonic push pulse to a biological tissue of a test object and transmission of a plurality of ultrasonic pulses for detection on the same sound ray in order to detect a shear wave generated in the biological tissue with the push pulse concerned are alternatively repeated, the program for controlling the ultrasonic probe such that the ultrasonic pulses for detection are transmitted on the sound ray until the shear wave is detected at a predetermined number of detection points on the sound ray that the ultrasonic pulses for detection are transmitted.

12. The ultrasonic diagnostic device defined in claim 1, wherein the processor controls the ultrasonic probe by the program such that after the shear wave has been detected at the predetermined number of detection points on the sound ray that the ultrasonic pulses for detection are transmitted, transmission of the plurality of the ultrasonic pulses for detection is started on a sound ray that is different from the sound ray.

13. The ultrasonic diagnostic device defined in claim 2, wherein the processor controls the ultrasonic probe by the program such that after the shear wave has been detected at the predetermined number of detection points on the sound ray that the ultrasonic pulses for detection are transmitted and before transmission of the ultrasonic pulses for detection on the sound ray that is different from the sound ray is started, the push pulse is transmitted.

14. The ultrasonic diagnostic device defined in claim 1, comprising:
a processor that executes a program for a measured value calculation function for calculating a measured value relevant to an elasticity of the biological tissue on the basis of an echo signal of the ultrasonic pulse for detection.

15. The ultrasonic diagnostic device defined in claim 2, comprising: a processor that executes a program for a measured value calculation function for calculating a measured value relevant to an elasticity of the biological tissue on the basis of an echo signal of the ultrasonic pulse for detection.

16. The ultrasonic diagnostic device defined in claim 3, comprising:
a processor that executes a program for a measured value calculation function for calculating a measured value relevant to an elasticity of the biological tissue on the basis of an echo signal of the ultrasonic pulse for detection.

17. The ultrasonic diagnostic device defined in claim 4, comprising:
a display unit on which a two-dimensional elastic image that has a display format according to the measured value is to be displayed.

18. The ultrasonic diagnostic device defined in claim 5, comprising:
a display unit on which a two-dimensional elastic image that has a display format according to the measured value is to be displayed.

19. The ultrasonic diagnostic device defined in claim 6, comprising:
a display unit on which a two-dimensional elastic image that has a display format according to the measured value is to be displayed.

20. The ultrasonic diagnostic device defined in claim 7, wherein the ultrasonic pulse for detection is transmitted to a two-dimensional region in which the two-dimensional elastic image is to be displayed.

21. The ultrasonic diagnostic device defined in claim 8, wherein the ultrasonic pulse for detection is transmitted to a two-dimensional region in which the two-dimensional elastic image is to be displayed.

22. The ultrasonic diagnostic device defined in claim 9, wherein the ultrasonic pulse for detection is transmitted to a two-dimensional region in which the two-dimensional elastic image is to be displayed.

23. The ultrasonic diagnostic device defined in claim 10, wherein alternate transmission of the push pulse and the ultrasonic pulse for detection corresponding to the push pulse concerned is repeated such that the ultrasonic pulses for detection are transmitted onto the different sound rays in the two-dimensional region.

24. The ultrasonic diagnostic device defined in claim 11, wherein alternate transmission of the push pulse and the ultrasonic pulse for detection corresponding to the push pulse concerned is repeated such that the ultrasonic pulses for detection are transmitted onto the different sound rays in the two-dimensional region.

25. The ultrasonic diagnostic device defined in claim 12, wherein alternate transmission of the push pulse and the ultrasonic pulse for detection corresponding to the push pulse concerned is repeated such that the ultrasonic pulses for detection are transmitted onto the different sound rays in the two-dimensional region.

26. The ultrasonic diagnostic device defined in claim 4, wherein the measured value calculation function is a propagation velocity calculation function for calculating a propagation velocity of the shear wave on the basis of the echo signal of the ultrasonic pulse for detection.

27. The ultrasonic diagnostic device defined in claim 5, wherein the measured value calculation function is a propagation velocity calculation function for calculating a propagation velocity of the shear wave on the basis of the echo signal of the ultrasonic pulse for detection.

28. The ultrasonic diagnostic device defined in claim 6, wherein the measured value calculation function is a propagation velocity calculation function for calculating a propagation velocity of the shear wave on the basis of the echo signal of the ultrasonic pulse for detection.

29. The ultrasonic diagnostic device defined in claim 4, wherein the measured value calculation function is an elasticity value calculation function for calculating an elasticity value of the biological tissue on the basis of the propagation velocity of the shear elastic wave calculated on the basis of the echo signal of the ultrasonic pulse for detection.

30. The ultrasonic diagnostic device defined in claim 5, wherein the measured value calculation function is an elasticity value calculation function for calculating an elasticity value of the biological tissue on the basis of the propagation velocity of the shear elastic wave calculated on the basis of the echo signal of the ultrasonic pulse for detection.
